(19) United States
(12) Patent Application Publication
(10) Pub. No.: US 2006/0241178 A1
(43) Pub. Date: Oct. 26, 2006

(54) Compositions and methods for treating lung cancer

(75) Inventors: Fung-Lung Chung, Rockville, MD (US); C. Clifford Conaway, Mahopac, NY (US); Yang-Ming Yang, Mahopac, NY (US)

Correspondence Address: Mr. Timothy H. Van Dyke, 390 N. Orange Avenue, Suite 2500, Orlando, FL, 32801, US

(73) Assignee: ImmuneGuard LLC, Winter Springs, FL (US)

(21) Appl. No.: 10/541,256

(22) PCT Filed: Dec. 30, 2003
(86) PCT No.: PCT/US03/41785

§ 371 Date: Apr. 17, 2006

Related U.S. Application Data
(60) Provisional application No. 60/437,240, filed on Dec. 30, 2002.

(51) Int. Cl. A61K 38/05; A61K 31/198; A61K 31/26

(52) U.S. Cl. 514/514; 514/18; 514/562

(57) ABSTRACT

The invention is directed to methods and compositions of inhibiting lung tumorigenesis. Such method involves the administration of an isothiocyanate conjugate at the post-initiation stage of tumor growth, while avoiding the drawbacks of toxicity of the parent compounds.

This application claims the benefit of U.S. provisional application No. 60/437,240, filed Dec. 30, 2002, which is incorporated by reference herein in its entirety.

The present invention was made with government support under grant number CA46535 from the National Institutes of Health (National Cancer Institute). Accordingly, the U.S. Government may have certain rights in the invention.

### FIELD OF THE INVENTION

The present invention relates to the prevention and treatment of lung cancer with the administration of an isothiocyanate conjugate. Specifically, the invention relates to the administration of an isothiocyanate thiol conjugate at the postinitiation stages of tumor development, and to inhibit tumors that have developed.

### BACKGROUND OF THE INVENTION

Isothiocyanates (ITCs), occurring as glucosinolates in cruciferous vegetables, have been shown to have cancer chemopreventive activity in laboratory animals (Josephsson, Phytochemistry (Oxf.) 1967, 5:1617-1627; Sones et al., J. Sci. Food Agric. 1984, 35:712-720). Studies indicate that ITCs are versatile anti-carcinogenic compounds for various organ sites, including lung, esophagus, mammary gland, liver, small intestine, colon, pancreas, and bladder (Wattenberg, Carcinogenesis (Lond.) 1987, 8: 1971-1973; Morse et al., Cancer Res. 1989, 49: 2894-2897; Chung, Cancer Chemoprevention 1992, 227-245, CRC Press Inc.; Stoner et al., Cancer Res. 1991, 51: 2063-2068; Hecht, J. Cell Biochem. Suppl. 1995, 22: 195-209; Zhang et al., Cancer Res. 1994, 54 (Suppl.): 1976s-1981s; Nishikawa et al., Carcinogenesis 1996, 17: 1381-1384). The widely investigated mechanisms by which ITCs inhibit tumorigenesis are the inhibition of cytochrome P450s involved in the activation of carcinogens and/or the induction of the phase II detoxifying enzymes, including glutathione S-transferases, quinone reductase, and UDP-glucuronosyltransferases (Hecht, J. Cell Biochem. Suppl. 1995, 22: 195-209; Zhang et al., Cancer Res. 1994, 54 (Suppl.): 1976s-1981s; Yang et al., Cancer Res. 1994, 54 (Suppl.): 1982a-1986s).

Previous studies have shown that pretreatment with ITC-thiol conjugates, similar to that with parent ITCs, inhibits lung tumorigenesis induced by the tobacco carcinogen 4-(methyl nitrosamino)-1-(3-pyridyl)-1-butanone (NNK) (Jiao et al., Carcinogenesis 1997, 18: 2143-2147, 1997). Studies also suggest that thiol conjugates of ITCs exert their activities by releasing ITCs and thiols via deconjugation (Jiao et al., Chem. Res. Toxicol. 1996, 9: 932-938; Conaway et al., Chem. Res. Toxicol. 2001, 14:1170-1176).

In addition to the activities of ITCs on phase I and phase II enzymes, recent studies in cell culture have shown that ITCs and their conjugates induce apoptosis, a protective mechanism against neoplastic development in which genetically damaged or improperly divided cells are eliminated (Huang et al., Cancer Res. 1998, 58: 4102-4106; Garnet-Payrastre et al., Cancer Res. 2000, 60: 1426-33; Chen et al., J. Biol. Chem. 1998, 273: 1769-1775; Yu et al., Cancer Res. 1998, 58: 402-408; Xu et al., Biochem. Pharmacol. 2000, 60: 221-231). In vitro and in vivo studies have demonstrated that suppression of apoptosis is involved in tumor promotion caused by chemical agents. It has been reported that ITCs induce JNK (c-Jun NH₂-terminal kinase) activation in cultured cells, and that this activation is associated with induction of apoptosis. Other studies have demonstrated that phenethyl ITC (PEITC) induces p53 transactivation in a dose- and time-dependent manner in a mouse epidermal cell line with accompanying apoptosis. In contrast, PEITC did not induce apoptosis in p53 (−/−) mouse embryo fibroblasts, suggesting that a p53-mediated mechanism is involved in ITC-induced apoptosis (Huang et al., Cancer Res. 1998, 58: 4102-4106).

A recent study showed that the N-acetyl-L-cysteine (NAC) conjugates of ITCs given orally after the administration of azoxymethane inhibit aberrant crypt foci formation in the colon of F344 rats (Chung et al., Carcinogenesis 2000, 21: 2287-2291). Both PEITC and sulforaphane, in unconjugated format, inhibited foci formation independent of the timing of administration.

A series of L-cysteine (L-Cys), glutathione (GSH) and NAC conjugates of phenethyl (PEITC), benzyl (BITC), and 6-phenylhexyl isothiocyanate (PHITC) were studied in vitro for their inhibitory activity toward metabolic activation of the tobacco-specific NNK in mouse lung microsomes (Jiao et al., Carcinogenesis 1997, 18: 2143-2147, 1997). In the study, PEITC, PEITC-GSH, PEITC-NAC and PHITC-NAC were administered prior to exposure to NNK, and were investigated for chemopreventive activity. Results demonstrated that the conjugated ITCs were less potent than the ITCs. However, the ITC conjugates were less toxic to the animals, judging from weight loss. To achieve the same efficacy and toxicity effects, two to three more times of the conjugate were needed in comparison to the parent compound.

Prior to the present invention, no in vivo efficacy of ITC conjugates against lung tumorigenesis has been examined when the ITC conjugates were administered at the post-initiation stage. In fact, given that ITC functionality is blocked by the thiol compounds, ITC thiol conjugate compounds would likely not work if administered after initiation. Given the prior work demonstrating the tumor preventative effects of glucosinolates in cruciferous sprouts (U.S. Pat. Nos. 5,725,895, 5,968,567, and 5,968,505), and that ITCs, also naturally occurring in cruciferous vegetables, have been identified as having a special role in cancer prevention (Wattenberg, Carcinogenesis 1987, 8(12):1971-1973), there remained a need to determine possible benefits of ITC thiol conjugates.

### SUMMARY OF THE INVENTION

It has now been discovered in the present invention that administration of an ITC conjugate at the post-initiation stage of lung tumor development has antitumorigenic effects with lessened toxicity. The present invention thereby provides an advantageous method of the treatment of incipient cancer.

Accordingly, the invention provides a method of inhibiting lung tumorigenesis in a mammal in need thereof, comprising administering to the mammal an effective amount of a conjugate of an isothiocyanate at the post-initiation stages of tumor growth.

In specific embodiments, the isothiocyanate is selected from the group consisting of phenethyl isothiocyanate; benzyl isothiocyanate; methyl isothiocyanate; ethyl isothiocyanate; propyl isothiocyanate; isopropyl isothiocyanate; n-butyl isothiocyanate; t-butyl isothiocyanate; s-butyl isothiocyanate; pentyl isothiocyanate; hexyl isothiocyanate; heptyl isothiocyanate; octyl isothiocyanate; nonyl isothiocyanate; decyl isothiocyanate; undecane isothiocyanate; phenyl isothiocyanate; o-tolyl, isothiocyanate; 2-fluorophenyl isothiocyanate; 3-fluorophenyl isothiocyanate; 4-fluorophenyl isothiocyanate; 2-nitrophenyl isothiocyanate; 3-nitrophenyl isothiocyanate; 4-nitrophenyl isothiocyanate; 2-chlorophenyl isothiocyanate; 2-bromophenyl isothiocyanate; 3-chlorophenyl isothiocyanate; 3-bromophenyl isothiocyanate; 4-chlorophenyl isothiocyanate; 2,4-dichlorophenyl isothiocyanate; R-(+)-alpha-methylbenzyl isothiocyanate; S-(−)-alpha-methylbenzyl isothiocyanate; 3-isoprenyl-alpha,alpha-dimethylbenzyl isothiocyanate; trans-2-phenylcyclopropyl isothiocyanate; 1,3-bis(isothiocyanatomethyl)-benzene; 1,3-bis(1-isothiocyanato-1-methylethyl)benzene; 2ethylphenyl isothiocyanate; benzoyl isothiocyanate; 1-naphthyl isothiocyanate; benzoyl isothiocyanate; 4-bromophenyl isothiocyanate; 2-methoxyphenyl isothiocyanate; m-tolyl isothiocyanate; alpha, alpha, alpha-trifluoro-m-tolyl isothiocyanate; 3-fluorophenyl isothiocyanate; 3-chlorophenyl isothiocyanate; 3-bromophenyl isothiocyanate; 1,4-phenylene diisothiocyanate; 1-isothiocyanato-4-(trans-4-propylcyclohexyl)benzene; 1-(trans-4-hexylcyclohexyl)-4-isothiocyanatobenzene; 1-isothiocyanato-4-(trans-4-octylcyclohexyl) benzene; 2-methylbenzyl isothiocyanate; 2-chlorobenzo isothiocyanate; 3-chlorobenzo isothiocyanate; 4-chlorobenzo isothiocyanate; m-toluyl isothiocyanate; and p-toluyl isothiocyanate.

Preferably, the isothiocyanate is selected from the group consisting of phenethyl isothiocyanate, benzyl isothiocyanate, and sulforaphane.

In one embodiment, the isothiocyanate conjugate is a thiol conjugate. Preferably, the thiol is selected from the group consisting of L-Cys, Glutathione, and N-acetyl-L-cysteine conjugates. More preferably, the thiol is a N-acetyl-L-cysteine.

In particular embodiments, the mammals are human subjects. Exemplary human subjects are smokers, ex-smokers, workers exposed to second-hand smoke, or chemical plant workers.

In a preferred embodiment, the administration is oral in tablet or capsule dosage form. Preferably, the dosage form comprises 20-80 mg of the conjugate, to be administered 2 to 3 times daily.

In a specific embodiment, the tumor growth is malignant. In another embodiment, the tumor growth is non-malignant.

The invention also provides a method of inhibiting lung tumorigenesis in a human in need thereof, which method comprises oral administration of 20-80 mg capsules of PEITC-NAC, two to three times daily, at the post-initiation stages of tumor growth.

The invention also provides a method of inhibiting lung tumorigenesis in a mammal in need thereof, which method comprises administering to the mammal an effective amount of phenethyl isothiocyanate NAC conjugate at the post-initiation stages of cancer.

The invention also provides for a pharmaceutical formulation comprising an isothiocyanate conjugate and a pharmaceutically acceptable carrier. In specific embodiments, the pharmaceutically acceptable carrier is a binder for tabletting, a capsule, or a USP grade buffered solution. In a preferred embodiment, the isothiocyanate conjugate is selected from the group consisting of phenethyl isothiocyanate-NAC, benzyl isothiocyanate-NAC, and sulforaphane-NAC.

### DESCRIPTION OF THE DRAWING

FIG. 1. Schematic showing a proposed molecular mechanism for inhibition of lung tumorigenesis by ITC conjugates via apoptosis.

### DETAILED DESCRIPTION

The present invention relates, to the chemopreventive potential of ITC conjugates. The present invention is specifically directed to the thiol conjugates of ITCs. These include glutathione and cysteine conjugates.

In particular, the invention provides a method of inhibiting lung tumorigenesis in individuals at the post-initiation stages of tumor development by administering an amount of the ITC conjugate effective to inhibit or reduce lung tumorigenesis. The invention is based in part on the finding that NAC conjugates of two widely-occurring ITCs, BITC and PEITC, have chemopreventive efficacy when administered during the post-initiation phase of Benzo(a)pyrene (B(a)P)-induced lung tumorigenesis in A/J mice. The compounds also have reduced toxicity as compared to the parent compounds. The conjugate compounds are more stable than the parent compounds, and thus have longer shelf-life.

As used herein, “carcinogen” refers to any cancer causing agent, such as tobacco, second-hand smoke, and hazardous chemicals. Other carcinogens may include, but are not limited to, asbestos, air pollutants, coal dust, and industrial fumes.

As used herein, “tumorigenesis” refers to the development of tumors. The tumors of the present invention may be non-malignant as well as malignant. Adenocarcinomas are the most common cell type of cancer since they include almost all breast cancers, all colon cancers, all prostate cancers, and a fair percentage of lung cancers. In the present invention, treatment is likely to be directed to the treatment of solid lung tumors.

In a preferred embodiment, the present invention is directed to lung cancer and the treatment of lung tumors. Non-limiting examples of lung tumor include the two major types, small cell lung carcinoma (SCLC) and non-small cell lung carcinoma (NSCLC). SCLC expresses neuroendocrine markers, and generally metastasizes early to lymph nodes, brain, bones, and liver. NSCLC comprises the majority of the remaining lung tumor types, and includes adeno-carcinoma, squamous cell carcinoma, and large cell carcinoma. NSCLC is characterized by the presence of epithelial-like growth factor receptors on the cells, and is locally invasive.

As used herein, the term “post-initiation” refers to any time period after exposure to a carcinogen, when a selected population of the initiated cells begin abnormal growth. The first stage of the formation of cancer cells is the initiation stage. During this stage, cellular mutations result in a loss or gain of a particular function resulting in abnormal growth. This stage allows the cancerous cell to progress to tumor development. Subsequent stages are followed by a promotion stage, in which the mutated cells acquire traits associated with benign tumor cells, and eventually, these cells go into the progression stage, which results in the development of malignant tumors and metastasis. The present invention is specifically directed to inhibiting the promotion and progression stages, particularly the promotion stage.

### Isothiocyanate Conjugates

As used herein, “isothiocyanate” (ITC) refers to any compound having the formula,
R—N═C═S,
where R may be saturated or unsaturated, substituted or unsubstituted, or an aliphatic or aromatic group. Non-limiting examples of R include phenethyl, benzyl, methyl; ethyl; propyl; isopropyl; n-butyl; t-butyl; s-butyl; pentyl; hexyl; heptyl; octyl; nonyl; decyl; undecane; phenyl; o-tolyl; 2-fluorophenyl; 3-fluorophenyl; 4-fluorophenyl; 2-nitrophenyl; 3-nitrophenyl; 4-nitrophenyl; 2-chlorophenyl; 2-bromophenyl; 3-chlorophenyl; 3-bromophenyl; 4-chlorophenyl; 2,4-dichlorophenyl; R-(+)-alpha-methylbenzyl; S-(−)-alpha-methylbenzyl; 3-isoprenyl-alpha,alpha-dimethylbenzyl; trans-2-phenylcyclopropyl; (SCN)CH₂C₆H₄CH₂—; (SCN)CH(CH₃)CH₂—C₆H₄CH₂CH(CH₃)CH₂—; CH₃S(O)CH₂CH₂CH₂CH₂—; 2-ethylphenyl; benzoyl; 1-naphthyl; benzoyl; 4-bromophenyl; 2-methoxyphenyl; m-tolyl; alpha, alpha, alpha-trifluoro-m-tolyl; 3-fluorophenyl; 3-chlorophenyl; 3-bromophenyl; (SCN)C₆H₄—; (propylcyclohexyl)benzyl; (hexylcyclohexyl)benzyl; (octylcyclohexyl)benzyl; 2-methylbenzyl; 2-chlorobenzo; 3-chlorobenzo; 4-chlorobenzo; m-toluyl; and p-toluyl. Preferably, R is phenethyl or benzyl or CH₃S(O)CH₂CH₂CH₂CH₂—.

The ITC can be either isolated from natural sources or prepared by chemical synthesis. Natural sources of ITC include cruciferous vegetables such as horseradish, radishes, onions, mustards, alyssum, candytuft, cabbage, and broccoli (U.S. Pat. Nos. 5,725,895, 5,968,567, and 5,968,505).

Non-limiting examples of ITCs include phenethyl isothiocyanate, benzyl isothiocyanate, sulforaphane (SFN); methyl isothiocyanate; ethyl isothiocyanate; propyl isothiocyanate; isopropyl isothiocyanate; n-butyl isothiocyanate; t-butyl isothiocyanate; s-butyl isothiocyanate; pentyl isothiocyanate; hexyl isothiocyanate; heptyl isothiocyanate; octyl isothiocyanate; nonyl isothiocyanate; decyl isothiocyanate; undecane isothiocyanate; phenyl isothiocyanate; o-tolyl isothiocyanate; 2-fluorophenyl isothiocyanate; 3-fluorophenyl isothiocyanate; 4-fluorophenyl isothiocyanate; 2-nitrophenyl isothiocyanate; 3-nitrophenyl isothiocyanate; 4-nitrophenyl isothiocyanate; 2-chlorophenyl isothiocyanate; 2-bromophenyl isothiocyanate; 3-chlorophenyl isothiocyanate; 3-bromophenyl isothiocyanate; 4-chlorophenyl isothiocyanate; 2,4-dichlorophenyl isothiocyanate; R-(+)-alpha-methylbenzyl isothiocyanate; S-(−)-alpha-methylbenzyl isothiocyanate; 3-isoprenyl-alpha,alpha-dimethylbenzyl isothiocyanate; trans-2-phenylcyclopropyl isothiocyanate; 1,3-bis(isothiocyanatomethyl)-benzene; 1,3-bis(1-isothiocyanato-1-methylethyl)benzene; 2-ethylphenyl isothiocyanate; benzoyl isothiocyanate; 1-naphthyl isothiocyanate; benzoyl isothiocyanate; 4-bromophenyl isothiocyanate; 2-methoxyphenyl isothiocyanate; m-tolyl isothiocyanate; alpha, alpha, alpha-trifluoro-m-tolyl isothiocyanate; 3-fluorophenyl isothiocyanate; 3-chlorophenyl isothiocyanate; 3-bromophenyl isothiocyanate; 1,4-phenylene diisothiocyanate; 1-isothiocyanato-4-(trans-4-propylcyclohexyl)benzene; 1-(trans-4-hexylcyclohexyl)-4-isothiocyanatobenzene; 1-isothiocyanato-4-(trans-4-octylcyclohexyl) benzene; 2-methylbenzyl isothiocyanate; 2-chlorobenzo isothiocyanate; 3-chlorobenzo isothiocyanate; 4-chlorobenzo isothiocyanate; m-toluyl isothiocyanate; p-toluyl isothiocyanate and the like. Preferably, the isothiocyanate is phenethyl or benzyl isothiocyanate or sulforaphane.

### Conjugates

The entities to conjugate to the ITCs to form a conjugate of the present invention include any thiol group that can be substituted on the ITC, including but not limited to glutathione, N-acetylcysteine, cysteine, and methionine. The thiol conjugate, while often less potent than the parent compound, is less toxic and more stable. In specific embodiments, the thiol conjugates of ITC are L-Cys, glutathione, and N-acetyl-L-cysteine conjugates.

### Formulation

Solid unit dosage forms may be prepared by mixing the compound, salt or derivative of the present invention with a pharmaceutically acceptable carrier and any other desired additives. The mixture is typically mixed until a homogeneous mixture of the compound of the present invention and the carrier and any other desired additives are formed, i.e., until the compound is dispersed evenly throughout the composition. In a preferred embodiment, the present invention is formulated as a solid prepared in a capsule form.

Biodegradable polymers for controlling the release of the compound include, but are not limited to, polylactic acid, polyepsilon caprolactone, polyhydroxy butyric acid, polyorthoesters, polyacetals, polydihydro-pyrans, polyanhydrides, polycyanoacrylates, cross-linked or amphipathic block copolymers of hydrogels, cellulosic polymers, and polyacrylates.

For oral administration, the therapeutics can take the form of, for example, tablets or capsules prepared by conventional means with pharmaceutically acceptable excipients such as binding agents (e.g., pregelatinized maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets can be coated by methods well known in the art. The preparations can also contain buffer salts, flavoring, coloring and sweetening agents as appropriate.

The phrase “pharmaceutically acceptable” refers to molecular entities and compositions that are “generally regarded as safe”, e.g., that are physiologically tolerable and do not typically produce an allergic or similar untoward reaction, such as gastric upset, dizziness and the like, when administered to a human. Preferably, as used herein, the term “pharmaceutically acceptable” means approved by a regulatory agency of the Federal or a state government or listed in the U.S. Pharmacopeia or other generally recognized pharmacopeia for use in animals, and more particularly in humans.

The term “carrier” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Suitable pharmaceutical carriers are described in “Remington's Pharmaceutical Sciences” by E. W. Martin.

### Salts and Derivatives

Various pharmaceutically acceptable salts, ether derivatives, ester derivatives, acid derivatives, and aqueous solubility altering derivatives of the active compound also are encompassed by the present invention. The present invention further includes all individual enantiomers, diastereomers, racemates, and other isomer of the compound. The invention also includes all polymorphs and solvates, such as hydrates and those formed with organic solvents, of this compound. Such isomers, polymorphs, and solvates may be prepared by methods known in the art, such as by regiospecific and/or enantioselective synthesis and resolution, based on the disclosure provided herein.

Suitable salts of the compound include, but are not limited to, acid addition salts, such as those made with hydrochloric, hydrobromic, hydroiodic, perchloric, sulfuric, nitric, phosphoric, acetic, propionic, glycolic, lactic pyruvic, malonic, succinic, maleic, fumaric, malic, tartaric, citric, benzoic, carbonic cinnamic, mandelic, methanesulfonic, ethanesulfonic, hydroxyethanesulfonic, benezenesulfonic, p-toluene sulfonic, cyclohexanesulfamic, salicyclic, p-aminosalicylic, 2-phenoxybenzoic, and 2-acetoxybenzoic acid; salts made with saccharin; alkali metal salts, such as sodium and potassium salts; alkaline earth metal salts, such as calcium and magnesium salts; and salts formed with organic or inorganic ligands, such as quaternary ammonium salts.

Additional suitable salts include, but are not limited to, acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, oleate, pamoate (embonate), palmitate, pantothenate, phosphate/diphosphate, polygalacturonate, salicylate, stearate, sulfate, subacetate, succinate, tannate, tartrate, teoclate, tosylate, triethiodide and valerate salts of the compound of the present invention.

The present invention includes prodrugs of the compound of the present invention. Prodrugs include, but are not limited to, functional derivatives of isothiocyanates that are readily convertible in vivo into isothiocyanates. Conventional procedures for the selection and preparation of suitable prodrug derivatives are described, for example, in “Design of Prodrugs”, ed. H. Bundgaard, Elsevier, 1985.

### Administration

The unit dosage forms of the present invention are administered to a patient suffering from lung cancer, preferably a human being. The patient, may be classified, but need not be, a smoker. In a specific embodiment, the patient may be, for example, an ex-smoker or a second-hand smoker. In another embodiment, the patient may be a chemical plant worker.

In the present invention, the method involves administering to the patient an effective amount of the ITC conjugate in a dosage regimen comprising administering to the patient a dosage form comprising a 20-80 mg capsule, two to three times daily, during the post-initiation phase of lung cancer such that there is intervention after cell commitment to dysplasia. The ITC conjugate is preferably administered orally.

The dosage regimen (amount and interval) of the compound of the present invention may vary according to a variety of factors such as underlying disease states, the individual's condition, weight, sex and age, the mode and route of administration; the renal and hepatic function of the patient; and the particular compound thereof employed. A physician or veterinarian of ordinary skill can readily determine and prescribe the effective amount of the drug required to prevent, counter or arrest the progress of the condition. Optimal precision in achieving concentrations of drug within the range that yields efficacy without toxicity requires a regimen based on the kinetics of the drug's availability to target sites. This involves a consideration of the absorption, distribution, metabolism, and excretion of a drug.

### Clinical Benefits and Uses

The present invention provides an interventional step in the progression of lung cancer. The treatment regimen has promising avenues including the ability to inhibit the development of lung adenoma (benign), which are good indicators of later cancers, as well as lung adenocarcinomas (malignant).

Furthermore, the present invention lends to the discovery of mechanism-based chemopreventive agents for ex-smokers who remain at an increased risk of lung cancer even after smoking cessation. In addition, the present invention may be used in smokers at higher risks or perhaps carry over with family history of lung cancer, and also in workers exposed to asbestos and other pollutants which may increase the risk of lung cancer.

### EXAMPLES

The following Examples illustrate the invention, but are not limiting.

### Example 1

### Inhibition of Benzo(a)pyrene-induced Lung Tumorigenesis in A/J Mice by Dietary N-Acetylcysteine Conjugates of Benzyl and Phenethyl Isothiocyanates During the Postinitiation Phase is Associated with Activation of Mitogen-activated Protein Kinases and p53 Activity and Induction of Apoptosis

The present example demonstrates the chemopreventive efficacy of the NAC conjugates of phenethyl isothiocyanate and benzyl isothiocyanate administered after, rather than before, initiation of lung tumorigenesis in mice.

### Methods

Diets, Chemicals and Reagents: PEITC, BITC, and NAC were purchased from Aldrich (Milwaukee, Wis.). The NAC conjugates of BITC and PEITC were prepared using a previously published method (Jiao et al., Carcinogenesis, 18: 2143-2147, 1997). Purity was verified by proton NMR spectra and by high performance liquid chromatography (>98%). B(a)P (purity>97%) and cottonseed oil were obtained from Sigma (St. Louis, Mo.). Other reagents used were obtained from commercial sources at the highest purity available.

The ITC conjugates were incorporated (15 μmol/g diet) into AIN-76A diets (5% corn oil) by mixing with dextrose prior to diet preparation. The conjugates, 9.36 g BITC-NAC (15 mmol) or 9.79 g PEITC-NAC (15 mmol), were dissolved in 50 ml ethyl acetate, and then mixed with 200 g dextrose to ensure even coating of dextrose particles. After mixing with dextrose, the solvent was removed using a rotary evaporator and further dried using a vacuum pump (2-3 h). Diets were prepared in 1-2 kg batches, and were stored at 4° C. in a container purged with nitrogen. PEITC-NAC and BITC-NAC prepared this way were stable for at least one month. Stability was determined by extraction of 0.5 g portions of the diet with 2.5 ml methanol (2×). The methanol extracts were combined and a 1 ml aliquot was filtered using a 0.47 μm syringe filter. A 10 μl sample was then analyzed on HPLC (Jiao, et al., Chem. Res. Toxicol., 9: 932-938, 1996).

Tumor Bioassay: Female strain A mice (Jackson Laboratories, Bar Harbor, Me.) of four weeks of age were housed under quarantine in polycarbonate cages (5 mice/cage) and provided modified AIN 76A diet (5% corn oil) and acidified drinking water ad libitum. The mice were maintained on a 12 h light: 12 h dark regimen at 22±5° C. and 50±20% relative humidity. After one week, the mice were weighed, and distributed into four groups containing 30 to 35 mice on the basis of body weight. At seven weeks of age, the mice in groups 1-3 were gavaged with a single dose of 20 μmol B(a)P in 0.2 ml cottonseed oil; group 4 received the vehicle only. Two days after dosing with the carcinogen, diets containing BITC-NAC (15 μmol/g, Group 2) and PEITC-NAC (15 μmol/g, Group 3) were provided. Groups 1 (B[a]P control) and 4 (vehicle only) remained on the modified AIN-76A diet with 5% corn oil. Tap water was provided ad libitum during the course of the bioassay. Mice were observed daily; diets were replenished on the fourth day of the week, and completely replaced on the seventh day. Mice were weighed weekly for four weeks, then monthly and at termination. At 84 days after B(a)P dosing, four mice per group were killed (CO₂, cervical dislocation) to harvest lung tissues for molecular and immunohistochemical studies and to quantify lung adenomas, if present. At 140 days after B(a)P, the remaining mice in each group were killed. The number of lung tumors was recorded; lobes of lungs were placed in 10% phosphate buffered formalin for histological and immunohistochemical analysis. The remaining lung lobes were snap frozen in liquid nitrogen.

In the second bioassay, A/J mice of the same age from Jackson Laboratory were treated using an identical protocol and maintained under the same conditions as described above for 21 days only. Diet consumption was measured twice weekly and body weights were determined weekly until termination. Mice were sacrificed at termination for harvesting lung tissues to be used in molecular studies.

Mean tumor multiplicity and body weights at each time point were compared between groups using Student's T test.

In situ end-labeling (ISEL): Formalin fixed paraffin embedded sections from the lungs of mice of the four experimental groups were prepared. ISEL (or TUNEL, terminal deoxynucleotide transferase dUTP nick end-labeling) was performed using an apoptosis detection kit (Enzo Diagnostics, Farmingdale, N.Y.) according to the manufacturer's instructions with the following exceptions: (1) endogenous peroxidase was blocked using 3% hydrogen peroxide in methanol for 15 minutes; (2) labeling solution was made up of 45 μl label reagent, 0.3 μl (10 μ/μl) terminal deoxynucleotide transferase (TdT) and 4.7 μl sterile distilled water; (3) sections were incubated in a 37° C. water bath for 15 min; (4) the color reaction with 3,3′-diaminobenzidine (DAB) was completed in 10 min on a 37° C. heating block; and (5) counter staining was done with Gill's 2 hematoxylin (Shandon-Lipshaw, Pittsburgh, Pa.) at a 1:10 dilution for 30 seconds. Sections of liver were used as controls. Cells undergoing apoptosis identified by ISEL were counted under a microscope; a total of 1,500 cells of alveolar epithelium from 20-24 visual fields per slide were tallied. Three slides per treatment group were analyzed.

Western Blot Analysis: Western blot analysis was performed as described previously (Ganju et al., J. Biol. Chem. 273: 23169-23175, 1998). Briefly, total proteins were prepared from each group of pooled mouse lungs. Lung samples were removed and immediately placed in 1×PBS with 2 mM DTT, 0.1 mM EDTA, and a protease inhibitor cocktail. The samples were then immediately transferred and homogenized in a radioimmunoprecipitation assay (RIPA) buffer with the protease inhibitors, aprotinin (1 μg/ml), leupeptin (1 μg/ml), pepstatin (1 μm), phenylmethylsulfonyl fluoride (0.1 mM) and the phosphatase inhibitors Na₃VO₄ (1 mM) and NaF (1 mM). Samples were centrifuged at 16,500 g for 30 min at 4° C. The supernatants were collected as the total proteins. Equal amounts (30 μg) of the total proteins were boiled for 5 min in the presence of Laemmli sample buffer, loaded on each lane, and separated by 10% SDS-PAGE. The gels were then transferred to nitrocellulose membranes. Equal amounts of protein loading for each lane was checked by Ponceau (Sigma, St. Louis, Mo.) staining. The anti-JNK 1, anti-phospho-JNK ½, anti-Bax (Santa Cruz Biotechnology, Santa Cruz, Calif.), anti-p53, anti-p21^{WAF1/CIP1} (Oncogene Res. Prod., Cambridge, Mass.), anti-phospho-p38, anti-phospho-Erk ½, and anti-phosphor-p53: Ser6, Ser 9, Ser 15, Ser 20, Ser 392 (Cell Signalling, Beverly, Mass.) antibodies were diluted to their useful concentration according to commercial recommendations. Immunoreactive bands were detected with an enhanced chemiluminescence kit (ECL, Amersham Pharmacia Biotech, Piscataway, N.J.).

Electrophoretic Mobility Shift Assay (EMSA): Double-stranded oligonucleotides containing the consensus binding site for AP-1, mutated AP-1, NFκB, and mutated NFκB were purchased commercially (Santa Cruz Biotechnology, Santa Cruz, Calif.). EMSA was performed as described previously (Yang et al., Cell Growth & Differentiation, 12: 211-21, 2001). Briefly, all oligos were labeled with γ³²P-ATP (6000 Ci/mmol, Amersham Pharmacia Biotech, Piscataway, N.J.) using polynucleotide kinase (Promega, Madison, Wis.) according to standard procedures. The labeled DNA (0.4 ng, 4400 cpm) was incubated with 10 μg of total proteins for 10 nin at room temperature, in the presence of 1 μg of poly d(I)-d(C) oligomer (Boehringer Mannheim, Indianapolis, Ind.) and DNA-binding buffer. The complexes were then separated on a 7.5% polyacrylamide gel and autoradiographed.

### Results

Inhibition of Tumor Multiplicity: At termination of the bioassay, lung adenomas were quantified and expressed as tumor multiplicity (number of tumors/mouse). Mice in Group 1 treated with B(a)P and fed the control diet had 6.1±3.1 tumors/mouse. Mice in Groups 2 and 3 treated with B(a)P followed by feeding diets containing BITC- and PEITC-NAC developed only 3.7±2.9 and 3.4±2.7 tumors/mouse, corresponding to a 39 and 44% of tumor reduction, respectively (Table 1).

TABLE 1
The multiplicity and incidence of
lung adenoma in treatment groups

| | | | Tumor |
|---|---|---|---|
| | No. | Tumor multiplicity | Incidence |
| Treatment Group | Mice | (no. of tumors/mice) | (%) |
| 1. B(a)P | 23 | 6.13 ± 3.13^{a} | 96 |
| 2. B(a)P + BITC-NAC | 18 | 3.72 ± 2.90^{b} | 94 |
| 3. B(a)P + PEITC-NAC | 18 | 3.39 ± 2.71^{b} | 89 |
| 4. Untreated control | 18 | 0.11 ± 0.31 | 11 |

^{a}= mean ± SD
^{b}= P < 0.05, compared with positive control group

Animals were observed throughout the bioassay and showed no signs of toxicity. However, mice fed the diet containing ITC compounds gained less body weight than control mice. At termination, the average body weight of Groups 2 (22.9 g) and 3 (22.8 g) was approximately 10 to 12% lower than Groups 1 (25.8 g) and 4 (25.7 g). The food consumption records showed that during the bioassay all mice gained approximately 0.1 g of weight per gram of food consumed. The reduction in body weight gains in these groups were, therefore, consistent with the reduced food consumption of mice in Groups 2 and 3 compared with Groups 1 and 4. These results suggest that the reduction in body weight gains for mice in the ITC treated groups was mainly due to palatability.

Increase in Apoptotic rate: Apoptosis in lung tissues obtained 84 and 140 days after administration of ITC diets was determined by ISEL. Results showed that the apoptotic indices were elevated approximately 2-fold in the BITC- and PEITC-NAC treated groups at 84 days, just before the tumors appeared. Similar results were obtained from BITC-NAC and PEITC-NAC groups after 140 days, when tumors had developed. Non-tumorous lung tissue of these two groups had more than a 2-fold increase in apoptosis (2.4-fold for BITC-NAC and 2.3-fold for PEITC-NAC). Because alveolar epithelial cells are mostly quiescent, a two-fold increase of the apoptotic cells may critically result in reduction of tumor multiplicity.

Activation of MP kinase activity: To study whether the activities of MAP kinases were altered by dietary treatment of ITC-NAC compounds, the total proteins from the lungs of the controls and ITC-treated mice were isolated. The activities of JNK in the lysates were determined by Western blot analysis. The phosphorylation levels on Ser 185 and 188 of JNK 1 and 2, detected by the phospho-specific antibody, were increased in lung tissue of ITC-NAC treated mice obtained 21 days after B(a)P administration. B(a)P treated groups showed no significant change in JNK 1 and 2 phosphorylation levels from the untreated group. Groups treated with B(a)P plus BITC-NAC and PEITC-NAC showed an increase of JNK 1 (p46) phosphorylation levels, while JNK 2 (p54) phosphorylations were only slightly induced, compared with JNK 1. The total proteins isolated from NIH 3T3 cells 15 min after UV (20 J) treatment served as a positive control for phospho-JNK 1 and 2. Equal amounts of JNK 1 were expressed in all groups. The results indicate that ITCs induce JNK 1 phosphorylation, but not its expression. When the total proteins in mouse lung tissue obtained 140 days after B(a)P treatment were examined, similar results were obtained showing an approximately 2-3 fold increase of phosphorylation of JNK 1 and 2 by ITC-NAC conjugates treatment.

The same blots used for phospho-JNK were stripped and re-probed with anti-phospho-p38 antibody. p38 phosphorylation levels did not change in mice treated with B(a)P compared with the untreated mice in Group 4. However, the ITC-NAC treated groups 2 and 3 showed a significant increase in p38 phosphorylation. The UV-treated NIH 3T3 cells as a positive control showed a strong phospho-p38 band. Erk 1 and 2 activities were detected in the same blot. The mice treated with B(a)P fed the control diet had a low level of phospho-Erk 2 (p42), whereas the groups fed the diets containing ITC conjugates showed an elevated phosphorylation level of Erk 2. However, Erk 1 phosphorylations were barely detectable in all groups.

Activation of p53 phosphorylation: To investigate the possible role of p53 in apoptosis induced by BITC-NAC or PEITC-NAC, we analyzed the expression of p53 and its phosphorylation level in lungs obtained at termination of the bioassay by Western blot using specific antibodies. While treatment with ITC compounds did not cause apparent accumulation of p53 or change the level of p53 expression, the level of phosphorylation of p53 at Ser15 appeared to be enhanced. BITC-NAC caused only a moderate increase, whereas the PEITC-NAC treatment resulted in a stronger increase in the phosphorylation. The phosphorylation levels of p53 serine 6, 9, 20, and 392 were also assayed. Phosphorylation at serine 9, 20, and 392 was induced in the ITC-NAC treated groups when compared with the B(a)P treated (Group 1) and untreated groups (Group 4). The phosphorylation at Ser 6 remained unchanged.

Expression of p53 effector genes p21^{WAF1/CIP1} and Bax: The activation of p53 by phosphorylation is expected to enhance the expression of p21^{WAF1/CIP1} and Bax (Chen et al., Cancer Res. 1995, 55: 4257-4263; Zhan et al., Oncogene 1994, 9: 3743-3751). The proteins from lung homogenate used for the p53 analysis were also assayed for p21^{WAF1/CIP1} and Bax by Western blot analysis. Our results show that, indeed, the activation of p53 in the lung tissues of ITC-NAC treated groups was accompanied by an increase in expression of p21^{WAF1/CIP1} and Bax. p21^{WAF1/CIP1} expression in the ITC-NAC treated groups was significantly higher than that in Groups 1 and 4. Although the induction of Bax expression was not as strong as p21^{WAF1/CIP1}, it is still clear that the treated groups showed increased expression of Bax.

Activation of transcription factors AP-1 but not NFκB: The increase of AP-1 and NFκB binding activity has been demonstrated in cultured human colon cancer cells treated with BITC, and BITC is believed to be involved in the induction of phase II enzymes (Patten et al., Biochem. Biophys. Res. Commun. 1999, 257:149-155). To determine whether dietary ITC-NAC compounds affect the transcription of genes regulated by AP-1 and NFκB, total protein extracts were prepared from the lung tissue of mice from all four groups 21 days after B(a)P administration. The binding activity of these proteins to AP-1 and NFκB, as well as to CRE and SIE target sequences was determined using the Electrophoretic Mobility Shift Assay (EMSA). AP-1 binding activities were strongly induced by ITC-NAC treatments. However, NFκB binding activities in ITC-NAC treated groups were not significantly different from the control. The binding activity induced by PEITC-NAC is specific for the AP-1 sequence, as the addition of a 10× unlabeled AP-1 sequence completely abolished binding activity. Similarly, the sustained NFκB binding activity is specific for the NFκB target sequence because a 10× unlabeled NFκB sequence effectively competed with the binding activity of the proteins from the untreated control group. Furthermore, a 10× unlabeled mutant AP-1 or NFκB sequences and a 10× extra non-specific DNA sequence did not alter the binding activity of AP-1 or NFκB.

### Discussion

The discovery of agents with the potential to reduce the risk of lung cancers that are effective when administered after exposure to tobacco carcinogens is an important step towards chemoprevention of cancer in ex-smokers. There are only a limited number of such agents so far identified from animal bioassays, with little known regarding their mechanisms of action in vivo (Wattenberg, Cancer Res. 1996, 56:5132-5135). The present study demonstrates for the first time that the NAC conjugates of BITC and PEITC can inhibit B(a)P-induced lung tumorigenesis during the post-initiation stages in vivo, at the same time, shedding light on the molecular mechanisms of inhibition in vivo by these agents.

In the tumor bioassay, body weight disparities between the groups fed diets containing ITC compounds and the control groups were noted. These differences were probably caused by the reduction of food consumption in the treated groups due to palatability, as indicated by the food consumption data. It raises questions as to whether the inhibition of lung tumorigenesis is a result of lowered caloric intake. Several lines of evidence suggest that this is not the case. First, little is known on the relationship of caloric restriction and lung tumorigenesis, but the extent of the decreases in body weight gain is probably too small to cause such a sizable reduction in tumor multiplicity based on published data for some other organ sites (Klufeld et al., J. Nutr. 1989, 119:286-291). Secondly, while it is known that caloric restriction could influence gene expression (Kritchevsky, Toxicol. Sci. 1999, 52:13-16), the molecular responses characterized in this study seem to be opposite to those found in animals on a caloric restricted diet. Without intending to limit the present invention to any particular theory not specifically recited in the claims, for example, it is believed that our studies showed that JNK 1, p38 and Erk 1 phosphorylation levels were induced by ITC compounds after 21 days of treatment, and AP1 activity was also strongly induced. Liu et al. reported that caloric restriction inhibits TPA induced AP1 binding activity, and also inhibit TPA-induced Erk activity, but not p38 and JNK in the epidermis of SENCAR mice (Liu et al., Carcinogenesis 2001, 22:607-612). Furthermore, our results demonstrated an induction of p53, p21, or Bax gene, yet others have shown that p53 phosphorylation and p27, p21, and p16 expression are not effected by caloric restriction in F344 rats (Piplin et al., Mechanisms Ageing Dev. 1997, 97:15-34).

Numerous studies in cell culture have shown that ITCs induce MAP kinase activity, AP-1, NFκB activity and p53 activity (Huang et al., Cancer Res. 1998, 58:4102-4106; Garnet-Payastre, et al., Cancer Res. 2000, 60:1426-1433; Chen et al., J. Biol. Chem. 1998, 273:1769-1775; Yu et al., Cancer Res. 1998, 58:402-408; Patten et al., Biochem. Biophys. Res. Commun. 1999, 257:149-155). This study is the first to demonstrate that oral administration of ITC compounds at the doses that inhibit lung tumorigenesis induces MAP kinase phosphorylation, AP-1 binding activity, and p53 activity in the target tissue of tumor inhibition. Compared to the results obtained from studies in cell culture, the activation of JNK activity in mouse lung was less pronounced, while the induction of the AP-1 activity was comparable. There were no detectable changes in NFκB binding activities in the mouse lung following treatments. This lack of activity suggests greater effectiveness for these compounds than what would have been expected from the in vitro work. The differences in molecular responses between in vitro and in vivo may be due to factors such as the concentrations of ITCs in culture medium versus tissue, cell-specific responses to ITCs, and/or uptake and metabolism in vivo. The activation of p53 is known to play a key role in the protection against tumorigenesis. Consistent with this, it is shown that BITC-NAC and PEITC-NAC activated p53 activity in mouse lungs by inducing phosphorylation and, subsequently, induced the expression of its effector genes: Bax and p21^{WAF1/CIP1}. However, questions regarding how these ITC compounds activate p53 phosphorylation still remain to be investigated. Taken together, dietary ITC conjugates induce molecular responses in mouse lung similar to those seen in ITC-treated cells in vitro, supporting the contention that the effects seen in lung are caused by ITCs released by deconjugation.

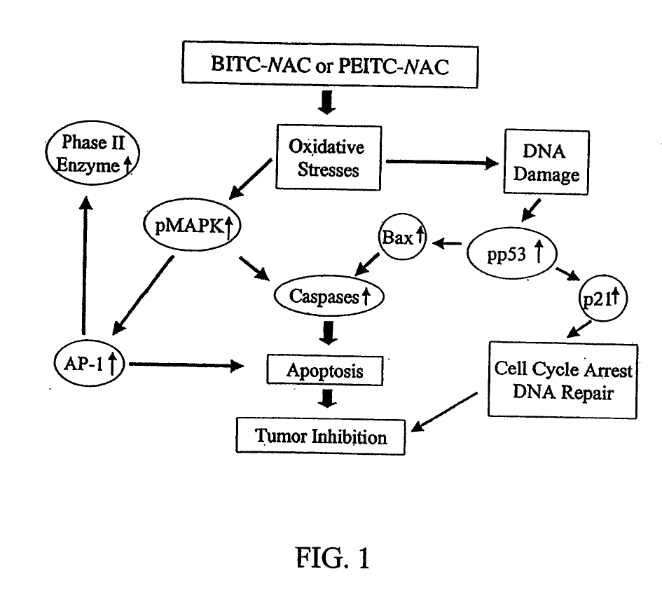

The cellular and molecular responses in mouse lungs after treatment with BITC-NAC and PEITC-NAC are known to be associated with oxidative stress. Although these compounds have not been shown specifically to cause oxidative DNA damage, some ITCs are cytotoxic, weakly mutagenic, and can stimulate lipid peroxidation in cultured cells (Kassie et al., Mutagenesis 1999, 14:595-603; Kassie et al., Chemico-Biol. Interact. 2000, 127:163-180). It is possible that ITC conjugates generated by deconjugation may cause oxidative DNA damage by depleting GSH and/or an alteration of the redox potential in lung cells by NAC (Liu et al., Cancer Res. 1998, 58:1723-1729; Sato et al., J. Immun. 1995, 154:3194-3203). To respond to the changes in oxidative status, it is believed that lung cells may go into apoptosis through activation of MAP kinases and p53. A proposed molecular mechanism for the inhibition of lung tumorigenesis by ITC conjugates via apoptosis is shown in FIG. 1. Clearly, more studies are needed to substantiate this mechanism. Regardless of the biochemical nature of the cellular stress caused by them, these ITC compounds are apparently not sufficient to induce tumorigenesis, as all of our previous bioassays in A/J mice or F344 rats showed that ITCs administered alone did not induce lung tumors (Jiao et al., Carcinogenesis 1997, 18: 2143-2147; Chung, Exp. Lung Res. 2001, 27:319-330). The results of this study show that administration of ITC conjugates in the diet during the post-initiation stages of B(a)P-induced lung tumorigenesis can elicit a series of stress-related molecular responses leading to cell death, which ultimately is manifested in the reduction of lung tumor formation.

### Example 2

### Study on Anti-Progression of Lung Tumorigenesis

The present example tests the ability of isothiocyanate thiol conjugates to inhibit the progression of lung tumorigenesis. A/J mice treated with tobacco carcinogens constituted the animal model for this study.

### Methods

The test compounds were given in the diet on week 21 after beginning of a weekly dose of a mixture of NNK/B(a)P: 3 μmol each (621 μg NNK and 756 μg B(a)P) in 0.1 ml cottonseed oil for eight weeks (a total of 8 doses) (groups 1 to 10); only vehicle (cottonseed oil) for groups 11 to 15. Animals were then given experimental diet for 20 weeks until week 40. Four mice in groups 1, 2, 4, 6, 8, and 10 (see Table 1) were sacrificed on weeks 20, 38, and 40. The bioassay was terminated on week 52. If sufficient numbers of tumors were developed in mice killed on week 40, the bioassay was terminated at week 40. Tumors (adenomas and carcinomas) were quantified by multiplicity and incidence. At sacrifice, livers and lungs were harvested and the tissues rinsed in autoclaved PBS, stored in labeled foil, and snap frozen in liquid nitrogen. These tissues both tumorous and non-tumorous were processed by histology for biomarker studies (TUNEL, PCNA,p53, MAO kinases, etc.). Lungs were examined by histology at termination (52 week or 40 week) for adenomas and carcinomas).

TABLE 1

| | Experimental | No. | Dosage per | Dose | Method |
|---|---|---|---|---|---|
| | Group | Animals | Animal | Frequency | Administ. |
| 1 | NNK + | 39 | 3 + 3 umol/— | Once/week | i.g./— |
| | B(a)P | | | (8wks) | |
| 2 | NNK + | 32 | 3 + 3 umol/3 | Once/week | i.g./diet |
| | B(a)P/ | | mmol/kg diet | (8wks)/daily | |
| | PEITC/High | | | | |
| 3 | NNK + | 20 | 3 + 3 umol/1.5 | Once/week | i.g./diet |
| | B(a)P/ | | mmol/kg diet | (8wks)/daily | |
| | PEITC/Low | | | | |
| 4 | NNK + | 32 | 3 + 3 umol/3 | Once/week | i.g./diet |
| | B(a)P/ | | mmol/kg diet | (8wks)/daily | |
| | SFN/High | | | | |
| 5 | NNK + | 20 | 3 + 3 umol/1.5 | Once/week | i.g./diet |
| | B(a)P/ | | mmol/kg diet | (8wks)/daily | |
| | SFN/Low | | | | |
| 6 | NNK + | 32 | 3 + 3 umol/8 | Once/week | i.g./diet |
| | B(a)P/ | | mmol/kg diet | (8wks)/daily | |
| | PEITC- | | | | |
| | Nac/High | | | | |
| 7 | NNK + | 20 | 3 + 3 umol/4 | Once/week | i.g./diet |
| | B(a)P/ | | mmol/kg diet | (8wks)/daily | |
| | PEITC- | | | | |
| | Nac/Low | | | | |
| 8 | NNK + | 32 | 3 + 3 umol/8 | Once/week | i.g./diet |
| | B(a)P/SFN- | | mmol/kg diet | (8wks)/daily | |
| | Nac/High | | | | |
| 9 | NNK + | 20 | 3 + 3 umol/4 | Once/week | i.g./diet |
| | B(a)P/SFN- | | mmol/kg diet | (8wks)/daily | |
| | Nac/Low | | | | |
| 10 | NNK + | 32 | 3 + 3 umol/8 | Once/week | i.g./diet |
| | B(a)P/Nac | | mmol/kg diet | (8wks)/daily | |
| 11 | Vehicle/ | 5 | 3 + 3 umol/3 | Once/week/ | —/diet |
| | PEITC | | mmol/kg diet | daily | |
| 12 | Vehicle/ | 5 | 3 + 3 umol/3 | Once/week/ | —/diet |
| | SFN | | mmol/kg diet | daily | |
| 13 | Vehicle/ | 5 | 3 + 3 umol/8 | Once/week/ | —/diet |
| | PEITC-NAC | | mmol/kg diet | daily | |
| 14 | Vehicle/ | 5 | 3 + 3 umol/8 | Once/week/ | —/diet |
| | SFN-NAC | | mmol/kg diet | daily | |
| 15 | Vehicle/ | 5 | 3 + 3 umol/8 | Once/week/ | —/diet |
| | NAC | | mmol/kg diet | daily | |
| 16 | Control | 5 | —/— | Once/week/ | —/— |
| | | | | daily | |

### Results

Preliminary results are provided below in Table 2.

TABLE 2

| | | Adenoma | | Adenocarcinoma | | |
|---|---|---|---|---|---|---|
| | | Multiplicity | | Multiplicity | Incidence | Overall % |
| Group No. | | mean ± SD | Incidence (%) | mean ± SD | (%) | Survival** |
| 1. | Control (n = 36) | 9.64 ± 5.29 | 36/36 (100%) | 1.06 ± 1.54 | 15/36 (42%) | 29.8% |
| 2. | PEITC-H (n = 32) | 8.81 ± 3.96 | 31/32 (96.90%) | 0.38* ± 0.94 | 6/32 (19%)* | 21.10% |
| 3. | PEITC-L (n = 20) | 7.25 ± 4.43 | 19/20 (95%) | 1.15 ± 1.85 | 7/20 (35%) | |
| 4. | SFN-H (n = 32) | 8.84 ± 5.72 | 30/32 (93.8%) | 0.84 ± 1.48 | 10/32 (31.3%) | |
| 5. | SFN-L (n = 20) | 9.25 ± 4.15 | 20/20 (100%) | 0.30 ± 0.64 | 4/20 (20%) | |
| 6. | PEITC-NAC H (n = 31) | 8.19 ± 3.99 | 30/31 (96.8%) | 0.52 ± 1.50 | 5/31 (16.1%) | |
| 7. | PEITC-NAC L (n = 19) | 7.58 ± 3.88 | 17/19 (89.47%) | 0.53 ± 1.14 | 4/19 (21.05%) | |
| 8. | SFN-NAC H (n = 31) | 9.06 ± 5.35 | 31/31 (100%) | 0.42 ± 1.01 | 5/31 (16.13%) | |
| 9. | SFN-NAC L (n = 19) | 8.53 ± 4.32 | 18/19 (94.74%) | 0.32 ± 0.92 | 2/19 (10.53%) | |
| 10. | NAC (n = 8) | 6/70 ± 4.47 | 8/8 (100%) | 1.10 ± 1.51 | 4/8 (50%) | |

*P < 0.05 compared to control after adjustment for survival time.
**Adjusted for scheduled sacrifices.

### Discussion

Isothiocyanates (ITCs) and N-acetylcysteine conjugates of isothiocyanates (ITC-NACs) inhibit lung adenoma formation induced in female A/J mice by tobacco carcinogens at initiation and post-initiation stages (Conaway et al., Current Drug Metab. 2002, 3: 233-255). In this study, the potential protective effects of ITCs and ITC-NACs against progression of lung adenomas to malignant tumors were investigated. Lung tumors were induced in A/J mice with 3 μmol B(a)P plus 3 μmol NNK (gavage once/wk, 8 wks); mice were maintained on AIN-76A semi-purified diets. At 20 weeks after the final treatment, lung adenomas (16.7±7.4 tumors/mouse) appeared in the treated mice examined. Diets containing PEITC (phenethyl ITC) or SFN (sulforaphane), each at 3 mmol/kg and 1.5 mmol/kg diet; PEITC-NAC or SFN-NAC at 8 mmol/kg and 4 mmol/kg diet; and NAC at 8 mmol/kg diet were then provided to groups of 28 mice (high dose) or 20 mice (low dose) during weeks 20 to 40 after initiation. Four mice in each high dose treatment group were killed during weeks 28 and 40 to monitor tumor incidence and progression; lung tissues were used for molecular studies. Remaining mice were killed at 40 weeks for histopathological examination. At termination, mean numbers of lung adenomas were reduced in all the ITC treatment groups compared with carcinogen control mice; the decreases were significant in groups fed PEITC-NAC, SFN-NAC, and NAC. Some mice also had developed forestomach masses. PEITC-NAC at 8 mmol/kg diet inhibited the incidence of adenocarcinoma/squamous carcinoma from 60% for initiated control group to 20%. Malignant tumor multiplicity was also reduced from 2.1 tumors/mouse in the initiated control group to 0.9 tumors/mouse in the SFN low dose and PEITC-NAC low dose groups; other treatment groups also showed reduced malignant tumor multiplicities to 1.0-1.6 tumors/mouse. To assess molecular events occurring during the treatments, lung RNA of mice treated with B(a)P+NNK/control diet and B(a)P+NNK/PEITC-NAC high dose diet was examined using a Mouse Cancer Pathway Finder Gene Array. In accordance with our previous observations, c-Jun expression (AP-1 pathway) and Akt pathway genes were up-regulated in the PEITC-NAC group. Findings involving other pathways will be presented. Our studies demonstrate the antiprogression activity for ITC-NACs and provide potential molecular bases for their chemopreventive action.

### Example 3

### N-acetylcysteine Conjugate of phenethyl isothiocyanate Selectively Enhance Apoptosis in Growth Stimulated Human Lung Adenoma

The present example demonstrates the role of AP-1 activity in PEITC-NAC induced apoptosis in human lung cells. Without intending to limit the present invention to any particular theory not specifically recited in the claims, it is believed that AP-1 activation has a dual role: (1) the induced activity of AP-1 prevented cell death and (2) the pre-conditional activation of AP-1 enhanced the amount of apoptosis induced by PEITC-NAC.

Methods

Cell Lines: The human lung adenocarcinoma cell line A549 was obtained from the American Type Culture Collection (ATCC) (Bethesda, Md.). It was found to be mycoplasma free, and maintained in DMEM supplemented with 10% fetal bovine serum (FBS). Cells were grown at 37° C. with 5% CO₂.

Reagents: The PEITC was obtained from Aldrich (Milwaukee, Wis.) and its N-acetylcysteine conjugate was synthesized in house by the Organic Synthesis Facility using the method described previously (Kassahun et al., Chem Res Toxicol 1997, 10(11):1228-33). Unless specified otherwise, the A549 cells were treated with 10 or 25 μM of PEITC-NAC. TPA (12-O-tetradecanoylphorbol-13-acetate) was purchased from Sigma (St. Louis, Mo.).

Electrophoretic Mobility Shift Assay (EMSA): Double-stranded oligonucleotides containing the consensus-binding site for AP-1 were purchased commercially (Santa Cruz Biotech). The oligos were labeled with γ-³²P-ATP (6000 Ci/mmol, Armeshem Pharmacia Biotech. Inc Piscataway, N.J.) using polynucleotide kinase (Promega, Madison, Wis.) according to standard procedures. The labeled DNA was incubated with 5 μg of total proteins (as specified in the Results section) for 10 min at room temperature, in the presence of 1 μg of poly d(I)-d(C) oligomer (Roche Molecular Biochemicals, Indianapolis, Ind.) and DNA-binding buffer as described previously (Yang et al., Cell Growth Differ 1993, 4(7):595-602). The complexes were then separated on a 7.5% polyacrylamide gel and autoradiographed.

Cell cycle analysis: A549 cells were harvested following 16 and 24 h treatment with various concentrations of PEITC-NAC by fixation in 70% ethanol. Cellular DNA was determined following staining with 1 μg/ml of 4,6-diamidino-2-ephynylindole (DAPI, Molecular Probes, Eugene, Oreg.) dissolved in PBS. Cellular blue fluorescence was measured using the Elite ESP flow cytometer/cell sorter (Coulter, Miami, Fla.) following excitation with a Ni/Cad UV light emitting laser. The data were collected and DNA histograms were deconvoluted using Multicycle software (Phoenix Flow Systems, San Diego, Calif.).

Transfection: The mammalian cell transfections were performed using a standard method previously described (Yang et al., Oncogene 1996, 12:2223-2233). 20 μg of pMEX MTH TAM67 plasmid, pMEX MTH-jun plasmid or the parent pMEX MTH plasmid (Freemerman et al., Mol. Pharmacol. 1996, 49:788-795) were transfected to 5×106 A549 cells using electroporation with 230 volts, 960 mF (BTX electroporation system) in 1× HEPES Buffer. After transfection, cells were then maintained in normal growth medium for 24 h, followed by the addition of G418 (800 μg/ml). For selection of stable neomycin-resistant transfectants, the cells were cultured in G418 selection medium for 10 days, then maintained in medium with 400 μg/ml G418. Three transfected cell lines were generated: (1) A549/c-jun was transfected with wild type c-jun; (2) A549/TAM67 were transfected with TAM67, the dominant negative mutant c-jun; and (3) A549/control-vector were transfected with the empty vector, pMEX MTH.

Northern Blot Analysis: RNA extraction, electrophoresis, gel transfer to nylon membranes and blot hybridization was performed as described previously (Yang et al., Cell Growth Differ 2001, 12(4):211-21). The blots were washed twice at 65° C. in 2×SSC and 0.1% SDS for 15 min. The c-jun cDNA insert (1.2 kb) of the pMEX MTH-jun plasmid was excised as a probe for hybridization and labeled by incorporation of α-³²P-dCTP (6000 Ci/mmol, Armeshem) into the c-jun sequence using a Random Primed DNA Labeling Kit (Roche Molecular Biochemicals). The amount of RNA loaded was monitored using 28 S and 18 S ribosomal RNA stained by ethidium bromide.

Analysis of DNA fragmentation: To confirm the appearance of apoptotic cells, nuclear DNA fragmentation was analyzed by agarose gel electrophoresis (Gong et al., Anal Biochem 1994, 218(2):314-9). The ethanol fixed cells were centrifuged at 800 g for 5 min, the cell pellets were resuspended in 40 μl of phosphate-citrate buffer, consisting of 192 parts of 0.2 M Na₂HPO₄ and 8 parts of 0.1 M citric acid (pH 7.8), at room temperature, for at least 30 min. After centrifugation at 1000 g for 5 min, the supernatant was transferred to new tubes and concentrated in a Speed Vac concentrator. A 3 μl aliquot of 0.25% NP-40 (v/v in H₂O) and 3 μl of RNase A solution (1 mg/ml) were added. After 30 min incubation at 37° C., 3 μl of proteinase K solution (1 mg/ml) was added and the extract was incubated for an additional 30 min at 37° C. After adding the loading buffer, the extract was subjected to electrophoresis on 0.8% agarose gel. The presence of the characteristic “ladder” pattern of discontinuous DNA fragments was visualized by ethidium bromide staining.

Annexin V apoptotic cell staining: The three transfected A549 cell lines, A549/control-vector (A, B), A549/c-jun (C, D), and A549/TAM67 (E, F), treated with 25 μM PEITC-NAC for 16 h. Three slides, A549/control-vector was on slide 1 and two photos (A and B) were taken from slide 1; A549/c-jun was on slide 2 and photos C and D taken from slide 2; A549/TAM67 was on slide. 3 and photos E and F taken from slide 3, were stained with Annexin V-Cy3 Apoptosis Detection Kit (Sigma, St. Louis, Mo.), which provides annexin V and 6-carboxyfluorescein diacetate dual staining. The protocol was followed according to the manufacturer's recommendation.

Proteins Isolation: Cells were incubated in a 37° C. with 5% CO₂, followed by mock or treatments specified in the results section. Total proteins were then isolated using RIPA buffer. The protease inhibitors aprotinin (1 μg/ml), leupeptin (1 μg/ml), pepstatin (1 μg/ml), phenylmethylsulfonyl fluoride (0.1 mM) and the phosphatase inhibitor Na₃VO₄ (1 mM), NaF (1 mM) were added to all buffers. The protein concentrations were determined using the Coomassie Plus Protein Assay Reagent (Pierce, Rockford, Ill.), and aliquots of the proteins were stored at −80° C.

Western Blot Analysis: Western blot analysis was performed as described previously (Yang et al., Cancer Res 2002, 62(1):2-7). Briefly, total proteins were prepared from the A549 cells with sham treatment or PEITC-NAC stimulation. Fifty micrograms of the total proteins were boiled for 5 min in the presence of Laemmli sample buffer, and then separated by 10% sodium dodecyl sulfate-polyacrylamide gel electrophoresis (SDS-PAGE). The proteins on the gel were transferred to nitrocellulose membranes. The anti-PARP apoptotic fragments antibody (Cell Signaling Tech. Inc. Beverly, Mass.) was diluted 1:400 before use. Immunoreactive bands were detected with a chemiluminescence kit, ECL (Amersham).

Assay for single cell DNA strand breaks: A549 cells treated with or without TPA, PEITC-NAC or TPA plus PEITC-NAC were collected at the times specified in the text, cytocentrifuged and stained with 4′-6′diamidino-2-phenylindole (DAPI). The photographs were taken using an Olympus AX 70 microscope (Melville, N.Y.) with fluorescence and SPOT RT Slider digital image system (Diagnostic Instruments Inc., Sterling Heights, Mich.). Arrows indicated the typical apoptotic features of DNA strand breakage and nuclear fragmentation.

Detection of cell cycle phase-specific DNA strand breaks (“TUNEL”): A549 cells were fixed with 1% formaldehyde for 15 min and then permabilized by post-fixation in 70% ethanol. The presence of in situ DNA strand breaks, a characteristic feature of apoptosis was detected by labeling them with a fluorochrome-conjugated nucleotide in the reaction catalyzed by the terminal deoxynucleotidyl transferase; cellular DNA was counterstained with DAPI. The kit provided by Phoenix Flow Systems (San Diego, Calif.) was used in this assay. This method is also described in detail elsewhere (Gorczyca et al., Internat J. Oncol. 1992, 1:639-648). Green and red fluorescence of cells probed for DNA strand breaks and DNA content was measured using a FACScan flow cytometer (Becton Dickinson, San Jose, Calif.) with standard settings of green (strand breaks) and red (DNA) fluorescence detection. To calculate the percentage of apoptotic cells in respective phases of cell cycle, DNA content frequency histograms were deconvoluted using CELLQuest software.

### Results

PEITC-NAC induces AP-1 activity in A549 cells in a dose- and time-dependent manner. PEITC-NAC induced AP-1 activity in lung tissue of A/J mice at a dose that inhibited tumorogenesis (Yang et al., Cancer Res 2002, 62(1):2-7). To determine whether PEITC-NAC induces AP-1 activity in human lung cells, total proteins were isolated from A549 human lung cells treated with PEITC-NAC. The binding activity of these proteins to AP-1 target sequences was assayed with EMSA. After 24 hour (h) treatment, AP-1 activation was stimulated by PEITC-NAC at a concentration as low as 1 μM. A double AP-1 binding band appeared at 5 μM PEITC-NAC. The peak binding activity appeared at 10 μM treatment with a clear double band. Higher concentrations of PEITC-NAC (25-100 μM) reduced the AP-1 activity from the peak dose (10 μM), which is probably a reflection of cell death. At 100 μM PEITC-NAC, no lung cells appear to survive after 24 h. In fact, the cells treated with 25-50 μM of PEITC-NAC for 24 h were undergoing the apoptosis, and as a result, most of these cells did not fully respond to AP-1 induction. AP-1 activation appeared as early as 30 minutes after 10 μM PEITC-NAC treatment, a double band appeared at the 6 h time point, and the activity remained elevated for periods up to 24 h, where a clear double band again appeared under identical treatment conditions (10 μM PEITC-NAC for 24 h). The observed binding activity was specific for AP-1 element. It could be competed by unlabeled AP-1 probe, but not the non-specific DNA fragment or mutant AP-1 probe.

PEITC-NAC treatment induces apoptosis in A549 cells. To examine whether PEITC-NAC causes apoptosis in human lung cells, as it did as in lung of A/J mice, flow cytometry was performed on cells treated with 10 μM PEITC-NAC for 24 h. When compared with control cells, a distinct sub-G1 peak appeared, which represents 4.5% of the cells that were undergoing apoptosis. This result demonstrates that the 10 μM PEITC-NAC is able to induce apoptosis in a small fraction of cells without affecting the survival of the majority of cells.

Dual roles of AP-1 in PEITC-NAC induced apoptosis: survival and death. To determine the relationship of AP-1 and apoptosis induction by PEITC-NAC, pMEX MTH TAM67, a dominant negative c-jun construct, as well as its empty vector pMEX MTH and a full-length c-jun cDNA construct pMEX MTH-jun were transfected into A549 cells via electroporation. Twenty-four hours after transfection the cells were selected by G418 (800 μg/ml) for 10 days to generate cell lines of A549/vector-control, A549/TAM67, and A549/c-jun. A549/c-jun cell line over-expresses c-jun mRNA (1.35 kb), and the A549/TAM67 cell line expresses TAM67 (truncated c-jun mRNA, 0.95 kb). The binding activity to AP-1 element was elevated in A549/c-jun cell line and reduced in A549/TAM67 cell line.

To evaluate the effect of AP-1 induction in the apoptotic process, several techniques were employed. Morphology and DNA fragmentation of the three transfected cell lines were studied after 24 h treatment with 25 μM PEITC-NAC was studied. Compared with the control-vector, TAM67 transfectants demonstrated a reduced capability to survive after the treatment. After 24 h of treatment, while the majority of control-vector transfectants were still alive, most TAM67 transfectants had already died. Interestingly, the c-jun transfected cells, with the highest AP-1 activity, did not show an increased survival, as opposed to TAM67 transfected cells. On the contrary, the cells showed enhanced apoptosis when compared with the control-vector transfected cells.

In addition, Annexin V-Cy3 and 6-carboxyfluorescein dual staining were performed on the vector-control, TAM67, and c-jun transfected cell lines to demonstrate the membrane evidence of apoptosis. Annexin V binds to phosphatidylserine moieties that become exposed on the outer surface of the cell membrane at apoptosis, while 6-carboxyfluorescein (6-CFDA) staining is the marker for viable cells. This combination allows the detection of early apoptotic cells (annexin V positive, 6-CFDA positive), necrotic cells or late apoptotic cells (annexin V positive, 6-CFDA negative), and viable cells (annexin V negative, 6-CFDA positive). Cells with green stain (6-carboxyfluorescein) only are viable, with red stain (annexin V-Cy3) only are necrotic cells, and with both red and green stains are early apoptotic cells. The results demonstrated that elevated AP-1 activity is important for cell survival during apoptosis induced by PEITC-NAC. TAM67 transfectants, which lacks AP-1 inducibility due to the fact that dominant negative c-jun interferes with the binding of transcription factors to the AP-1 target sequence, had nearly no cell survival 20 h after treatment with 25 μM PEITC-NAC compared with vector-control transfectants, in which although apoptosis had already begin, dead cells were not predominant. Concurrent with morphological observations, c-jun transfected A549 cells had enhanced their apoptosis compared with the control-vector transfected cells. Those results indicate that cells with a higher background on AP-1 activity were more sensitive to PEITC-NAC with regard to induction of apoptosis. Thus, AP-1 demonstrated a dual role in PEITC-NAC induced apoptosis: in the instance that PEITC-NAC unable to induce AP-1 activity, as in TAM67 transfectants, the cells would lack of the capability for survival response; on the other hand, cells were more competent to PEITC-NAC induced apoptosis if AP-1 had been activated.

The cleavage of poly (ADP-ribose) polymerase (PARP) as a marker for apoptosis was detected by Western blot. PARP is one of the main cleavage targets of caspase-3. In human, cleavage occurs between Asp214 and Gly215, which separate the N-terminal DNA binding domain (24 kD) of PARP from its C-terminal catalytic domain (89 kD). Three transfected cell lines were evaluated after treatment with 25 μM PEITC-NAC for 3 or 24 h. Three hours after the treatment a very light 89 kD cleavage fragment showed in the control-vector transfectants, while TAM67 transfectants had a defined 89 kD band, and the most evident cleavage occurred in the c-jun transfectants. After 24 h, however, TAM67 transfectants revealed advanced aggressive protein degradation. At this time point control-vector and c-jun cDNA transfected cells maintained a significant levels of intact PARP protein, whereas, PARP in TAM67 transfectants was completely degraded. These results indicate that TAM67 transfected cells went through apoptosis or necrosis within 24 h after PEITC-NAC treatment.

Apoptosis induced by PEITC-NAC is selectively enhanced in promoted cells. Since A549 cells transfected with c-jun cDNA were demonstrated to have an increased potential for apoptosis, the responses of PEITC-NAC treatment on TPA-pretreated cells that had elevated AP-1 activity were investigated. TPA (100 nM) was added to A549 cells 12 h prior to addition of PEITC-NAC, which was added 24 h before photography of cells, protein isolation, or DAPI staining. Photographs of A549 cells treated with TPA and PEITC-NAC, compared with the cells treated with PEITC-NAC alone, had a much higher incidence of apoptosis, while cells treated with control vehicle or treated with TPA alone appear to be growing well. AP-1 binding activity was specifically induced in A549 cells treated with 100 nM TPA for 24 h. These cells were treated identically, but were stained with the DNA fluorochrome DAPI. The cells showed typical features of apoptosis, i.e., DNA strand breakage and nuclear fragmentation after PEITC-NAC or TPA plus PEITC-NAC treatment. These results indicate that apoptosis induced by PEITC-NAC was enhanced in the TPA-pre-treated cells.

Apoptosis induced by PEITC-NAC occurs predominantly in dividing cells. To reveal the mechanism that may be responsible for the enhancement of apoptosis in growth stimulated cells, the cell cycle phase specificity of PEITC-NAC-induced apoptosis was investigated. To this end, A549 cells were treated with 25 μM PEITC-NAC for 16 h or with 50 μM PEITC-NAC for 24 h. DNA strand breaks during apoptosis was detected by end-labeling, which was combined with analysis of the cellular DNA content. This method allows the correlation of apoptotic cells with the specific phase of the cell cycle. Sixteen hours after treatment with 25 μM PEITC-NAC, a substantial portion of the cells accumulate in G2M phase. In cells treated with 50 μM PEITC-NAC for 24 h, the G2M fraction was significantly reduced but was accompanied by a distinct population of apoptotic cells identified by incorporation of BrdUTP into DNA strand breaks at a position consistent with those cells having originated from G2M population. The slight shift to the left of the apoptotic population is a result of DNA degradation; small molecular weight DNA removed during cell washing decreases the total stainable DNA. Thus, actively growing cells compared with cells resting in G0-G1 phase, had an increased propensity for undergoing apoptosis following exposure to PEITC-NAC.

### Discussion

This study demonstrates that, similar to the observation in lung tissue of A/J mice with oral administration of PEITC-NAC, PEITC-NAC induced apoptosis accompanied induction of AP-1 binding activity in human lung cancer cells. The dual roles of AP-1 in maintenance of cell survival and enhancement of apoptosi were also characterized in A549 human lung adenocarcinoma cells undergoing apoptosis after treatment with PEITC-NAC.

C-jun expression and AP-1 activity associated with apoptosis is induced by various agents, such as ionizing radiation, SV40 T antigen, vitamin E succinate, anti-tumor drugs etc. (Goldstone et al., Oncogene 1994, 9(8), 2305-11; Ferrer et al., Neurosci Lett 1995, 202(1-2), 105-8; Chen et al., Virology 1998, 244(2), 521-9; and Qian et al., Oncogene 1997, 15(2), 223-30). It has been reported that c-Jun expression and its N-terminal phosphorylation can promote neuronal apoptosis (Ham et al., Biochem Pharmacol 2000, 60(8), 1015-21; Terwel et al., Neuroscience 2000, 96(2), 445-6; and Rubin, Br Med Bull 1997, 53(3), 617-31). Experiments with sympathetic neurons cultured in vitro, as well as with cerebellar granule neurons and differentiated PC12 cells, have demonstrated that JNK/c-Jun signaling can promote apoptosis following survival factor withdrawal. When c-Jun phosphorylation site mutants were expressed in cerebellar granule neurons, c-Jun phosphorylation was necessary for apoptosis. c-Jun[asp], a constitutively active c-Jun mutant in which the known and potential serine and threonine phosphoacceptor sites in transactivation domain have been mutated to aspartic acid, induces apoptosis under all conditions tested. In contrast, c-Jun[ala], which cannot be phosphorylated because the same sites have been mutated to alanine, blocks apoptosis caused by survival signal withdrawal. In non-neuronal tissues, however, the role of AP-1 activation in apoptosis was reported very differently (Behrens et al., Nat Genet 1999, 21(3), 326-9; Bossy-Wetzel et al., Embo J 1997, 16(7), 1695-709; and Wisdom et al., Embo J 1999, 18(1), 188-97). In apoptosis induced by H₂O₂ and certain antitumor agents, cells transfected with a dominant negative c-jun mutant (TAM67) exhibited delayed apoptosis and increased overall survival (Wang et al., Circ Res 1999, 85(5), 387-93). The studies in 3T3 mouse fibroblasts and human umbilical vein endothelial cells showed that increased c-Jun activity was sufficient to trigger apoptotic cell death (Bossy-Wetzel et al., Embo J 1997, 16(7), 1695-709; and Wang et al., Circ Res 1999, 85(5), 387-93). On the other hand, the anticancer drugs were reported to induce apoptosis in human acute leukemia cells without involvement of the induction of c-jun expression (Bullock et al., Clin Cancer Res 1995, 1(5), 559-64). Furthermore, it has demonstrated that c-Jun protects cells from UV-induced cell death and cooperates with NκKB in the prevention of apoptosis induced by tumor necrosis factor α (Wisdom et al., Embo J 1999, 18(1), 18.8-97). The observation that a A549 subline transfected with a dominant negative c-jun mutant (TAM67) had a reduced capability for survival during PEITC-NAC induced apoptosis is concordant with prior information that c-jun up-regulation in U937 cells represents a response to, rather than a cause of, events in apoptosis (Freemerman et al., Mol Pharmacol 1996, 49(5), 788-95). TAM67 transfected cells in the late stages of apoptosis or necrosis when control cells were in relatively early stage of apoptosis, so the results demonstrated that induction of AP-1 activity is necessary for cell survival under conditions of PEITC-NAC induced cell death. On the other hand, data from c-jun transfected cells indicate that if elevated AP-1 activity occurs as a pre-existing condition, either by over-expression of the c-Jun oncogene or by treatment with growth promoting agent TPA, PEITC-NAC had an enhanced apoptotic effect.

Certain agents derived from fruits and vegetables, such as all-trans-retinoic acid and grape seed proanthocyanidin, have an anti-apoptotic action that occurs through JNK and AP-1 activation (Moreno-Manzano et al., J Biol Chem 1999, 274(29), 20251-8; and Sato et al., Free Radic Biol Med 2001, 31(6), 729-37). Selenium compounds used as chemopreventive agents also induce apoptosis, but AP-1 activation seems to be leading to the apoptosis in that instance, since TAM67 transfected cells showed reduced apoptosis induced by selenodiglutathione (Ghose et al., Cancer Res 2001, 61(20), 7479-87). Induction of apoptosis by various ITCs, including PEITC, has been reported to be mediated by JNK (Chen et al., J Biol Chem 1998, 273(3), 1769-75; and Dong, Biofactors 2000, 12(1-4), 17-28). PEITC-NAC induced a clear stress-response in A549 cells. The stress-induced genes such as JNK (data not shown) and PARP (FIG. 6, vector-control 0 and 3 hour) were stimulated after treatment with PEITC-NAC. c-Jun is one of the direct substrates of JNK, and AP-1 activity was induced by PEITC-NAC. However, results from TAM67 transfectants demonstrate that AP-1 activation induced by PEITC-NAC is not the cause of apoptosis, since prevention of AP-1 transcriptional activation (by dominant negative inhibition) accelerates cell death. This study implies that JNK activation induced by PEITC-NAC must stimulate some apoptotic pathway independent of AP-1. Elevated phosphorylation level of p53 was demonstrated in the mouse lung tissue after 3 weeks of oral administration of PEITC-NAC (Yang et al., Cancer Res 2002, 62(1), 2-7). Huang et al. (Huang et al., Cancer Res 1998, 58(18), 4102-6) demonstrated p53 is essential for PEITC induced apoptosis, which implicated that p53 pathway could possibly be a good candidate as ITCs apoptotic pathway. Since JNK phosphorylated p53 been described in various p53 activation systems (She et al., Mol Carcinog 2002, 33(4), 244-50; Zhang et al., J Biol Chem 2002, 277(5), 3124-31; Fuchs et al., Proc Natl Acad Sci USA 1998, 95(18), 10541-6; and Adler et al., Proc Natl Acad Sci USA 1997, 94(5), 1686-91), our hypothesis is that PEITC-NAC induced JNK activity up-regulates the apoptotic pathway through p53, in the meantime JNK activates AP-1, which confers resistance to cell death. The fate of the cells, whether that of death or survival, is determined by the predominant pathway.

The phorbol esters are natural compounds have been for many years used as pharmacological tools. They mimic the action of the lipid second messenger diacylglycerol by activating PKC. Although phorbol esters induce apoptosis in certain cell lines (Li et al., Oncogene 1998, 17(22), 2915-20; and Giese et al., Biol Cell 1997, 89(2), 99-111), they are well known as a promoters for mitogenesis in most cells. It has been reported that TPA can protect HL-60 cells from taxol-induced apoptosis and can block fas receptor-induced apoptosis in Jurkat and U937 cells (Pae et al., Immunopharmacol Immunotoxicol 2000, 22(1), 61-73; Gomez-Angelats et al., J Biol Chem 2000, 275(26), 19609-19; and Sordet et al., Cell Death Differ 1999, 6(4), 351-61). Contrariwise, TPA in this study system showed a different effect from those systems. It did not cause apoptosis in A549 cells and did not protect A549 cells from PEITC-NAC induced apoptosis; it enhanced the apoptosis. After 100 nM TPA treatment, cells were actively growing and dividing and cell death was not observed. The observation that PEITC-NAC enhanced the apoptotic process in TPA-promoted A549 cells, combined with the results from c-jun oncogene transfected A549 cells, implies a unique role for ITCs as a chemopreventive agent, which selectively enhances apoptosis in promoted cells.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

Patents, patent applications, publications, product descriptions, and protocols are cited throughout this application, the disclosures of which are incorporated herein by reference in their entireties for all purposes.

1. A method of inhibiting lung tumorigenesis in a mammal in need thereof, which method comprises administering to the mammal an effective amount of a conjugate of an isothiocyanate at the post-initiation stages of tumor growth.

2. The method of claim 1 wherein the isothiocyanate is selected from the group consisting of phenethyl isothiocyanate; benzyl isothiocyanate; methyl isothiocyanate; ethyl isothiocyanate; propyl isothiocyanate; isopropyl isothiocyanate; n-butyl isothiocyanate; t-butyl isothiocyanate; s-butyl isothiocyanate; pentyl isothiocyanate; hexyl isothiocyanate; heptyl isothiocyanate; octyl isothiocyanate; nonyl isothiocyanate; decyl isothiocyanate; undecane isothiocyanate; phenyl isothiocyanate; o-tolyl isothiocyanate; 2-fluorophenyl isothiocyanate; 3-fluorophenyl isothiocyanate; 4-fluorophenyl isothiocyanate; 2-nitrophenyl isothiocyanate; 3-nitrophenyl isothiocyanate; 4-nitrophenyl isothiocyanate; 2-chlorophenyl isothiocyanate; 2-bromophenyl isothiocyanate; 3-chlorophenyl isothiocyanate; 3-bromophenyl isothiocyanate; 4-chlorophenyl isothiocyanate; 2,4-dichlorophenyl isothiocyanate; R-(+)-alpha-methylbenzyl isothiocyanate; S-(−)-alpha-methylbenzyl isothiocyanate; 3-isoprenyl-alpha,alpha-dimethylbenzyl isothiocyanate; trans-2-phenylcyclopropyl isothiocyanate; 1,3-bis(isothiocyanatomethyl)-benzene; 1,3-bis(1-isothiocyanato-1-methylethyl)benzene; 2-ethylphenyl isothiocyanate; benzoyl isothiocyanate; 1-naphthyl isothiocyanate; benzoyl isothiocyanate; 4-bromophenyl isothiocyanate; 2-methoxyphenyl isothiocyanate; m-tolyl isothiocyanate; alpha, alpha, alpha-trifluoro-m-tolyl isothiocyanate; 3-fluorophenyl isothiocyanate; 3-chlorophenyl isothiocyanate; 3-bromophenyl isothiocyanate; 1,4-phenylene diisothiocyanate; 1-isothiocyanato-4-(trans-4-propylcyclohexyl)benzene; 1-(trans-4-hexylcyclohexyl)-4-isothiocyanatobenzene; 1-isothiocyanato-4-(trans-4-octylcyclohexyl) benzene; 2-methylbenzyl isothiocyanate; 2-chlorobenzo isothiocyanate; 3-chlorobenzo isothiocyanate; 4-chlorobenzo isothiocyanate; m-toluyl isothiocyanate; and p-toluyl isothiocyanate.

3. The method of claim 1 wherein the isothiocyanate is selected from the group consisting of phenethyl isothiocyanate, benzyl isothiocyanate, and sulforaphane.

4. The method of claim 1 wherein the conjugate is a thiol conjugate.

5. The method of claim 4 wherein the thiol is selected from the group consisting of L-Cys, Glutathione, and N-acetyl-L-cysteine conjugates.

6. The method of claim 4 wherein the thiol is a N-acetyl-L-cysteine.

7. The method of claim 1 wherein the mammal is a human.

8. The method of claim 7, wherein the human is selected form the group consisting of smokers, ex-smokers, workers exposed to second-hand smoke, and chemical plant workers.

9. The method of claim 1 wherein the administration is oral.

10. The method of claim 1 wherein the conjugate is administered orally as a tablet or a capsule.

11. The method of claim 1 wherein the amount administered is 20-80 mg, two to three times daily.

12. The method of claim 1 wherein the tumor growth is malignant or non-malignant.

13. A method of inhibiting lung tumorigenesis in a human in need thereof, which method comprises oral administration of 20-80 mg capsules of PEITC-NAC, two to three times daily, at the post-initiation stages of tumor growth.

14. A method of inhibiting lung tumorigenesis in a mammal in need thereof, which method comprises administering to a human an effective amount of phenethyl isothiocyanate NAC conjugate at the post-initiation stages of cancer.

15. A pharmaceutical formulation comprising an isothiocyanate conjugate and a pharmaceutically acceptable carrier.

16. A pharmaceutical formulation of claim 15, wherein the pharmaceutically acceptable carrier is a USP grade buffered solution.

17. A pharmaceutical formulation of claim 15, wherein the isothiocyanate conjugate is selected from the group consisting of phenethyl isothiocyanate-NAC, benzyl isothiocyanate-NAC, and sulforaphane-NAC.
